(19) United States
(12) Patent Application Publication
(10) Pub. No.: US 2022/0370204 A1
(43) Pub. Date: Nov. 24, 2022

(54) METHOD TO BIOPRINT A PATIENT SPECIFIC BONE GRAFT

(71) Applicant: Michael J. HARTMAN, Lewisberry, PA (US)

(72) Inventor: Michael J. HARTMAN, Lewisberry, PA (US)

(21) Appl. No.: 17/741,159

(22) Filed: May 10, 2022

Related U.S. Application Data
(60) Provisional application No. 63/190,923, filed on May 20, 2021.

(51) Int. Cl. A61F 2/30; A61F 2/28; A61L 27/36; B33Y 10/00; B33Y 80/00; B33Y 50/00

(52) U.S. Cl. CPC A61F 2/30942; A61F 2/2846; A61L 27/3616; A61L 27/365; B33Y 10/00; B33Y 80/00; B33Y 50/00; A61F 2/2803; A61F 2002/2835; A61F 2002/30952; A61F 2002/30985; A61F 2002/4633

(57) ABSTRACT

A system or method for bioprinting bone graft provides obtaining an image of the patient's oral facial area, and viewed with the image viewing software. A restoratively driven dental implant treatment plan is created to restore the patient's missing dentition. The restoratively driven treatment plan is created. A physical exam, review of a patient's desires and expectations, review of imaging, acquisition and review of patient photographs and intraoral digital impressions. The imaging and digital impressions are aligned, via software to create a virtual representation. The anticipated final implant retained dentures, unitary implant crowns, or implant bridges, are planned to provide optimal esthetic and functional results. Dental implants are then planned for prosthetic anchors. Bone deficiencies are evaluated and if areas of boney deficiency are present, a patient specific bone graft is designed to restore said deficient areas. Once designed, it may be printed via additive manufacturing.

### CROSS-REFERENCE TO RELATED PATENT APPLICATIONS

This patent application claim the benefit of and priority to U.S. Provisional Patent Application Ser. No. 63/190,923 entitled “A METHOD TO BIOPRINT A PATIENT SPECIFIC BONE GRAFT”, filed May 20, 2021, which patent application is hereby incorporated by reference.

### BACKGROUND

The application generally relates to bone grafts. The application relates more specifically to methods for grafting bone material via bioprinting that is specific to a patient.

Large boney defects such as ones caused by trauma, congenital malformations, cancer, or tooth loss can be an esthetic and functional challenge for a surgeon to reconstruct. Currently, the gold standard for large boney defect reconstruction is an autogenous bone graft. An autogenous bone graft is harvested from a donor site on the patient with the afflicted defect. While acceptable results are obtained, the autogenous bone graft has disadvantages. These disadvantages include increased post-operative discomfort due to the additional surgical site, increased operative time, increased potential risks, and a sub optimal esthetic result due to the graft not being able to conform and repair the defect in a precise and accurate manner.

Bone regeneration occurs through three possible mechanisms: osteogenesis, osteoconduction, and osteoinduction. These mechanisms involve boney cells capable of producing new bone, cellular signals and growth factors which aid in new bone growth, and a scaffold on which new bone can grow. The ideal bone graft material would contain all of these mechanisms, be designed in a manner to accurately and precisely restore the boney defect, and be able to be fabricated prior to the reconstructive procedure without the need for additional donor site surgery. As used herein, to bioprint means to use 3D printing technology with materials that incorporate viable living cells to produce tissue for reconstructive bone graft surgery.

The method described below describes how a patient specific, three-dimensional, bone graft is designed and bioprinted to reconstruct boney defects. Immediately prior to surgery, the bone graft can be saturated with the patient's blood products or bone marrow aspirate. The patient specific bone graft fabricated by this method will satisfy the requirements for an ideal bone graft material.

### BRIEF SUMMARY OF THE INVENTION

The present invention describes a series of systems and methods to design and additively manufacture a patient specific bone graft. The present embodiments may be particularly useful in patient's afflicted with craniofacial congenital skeletal malformations, patients which have had ablative surgery due to cancerous growths, and patients who have had alveolar bone loss due to tooth loss who are now desiring dental implant rehabilitation.

In one embodiment a method is disclosed for bioprinting a three-dimensional bone graft for a patient comprising. The method includes acquiring an image of an affected area of a boney defect; reviewing the image and generating a diagnosis and a treatment plan to restore the boney defect; segmenting a region of the image having the boney defect; designing a bone graft based on the boney defect; printing the bone graft using a biomaterial; obtaining a platelet based composition derived from a patient undergoing the implant treatment; saturating the bone graft with the platelet based composition; and inserting the bone graft; and fixating the bone graft to the affected area.

Another embodiment discloses a method for bioprinting a three-dimensional bone graft for a patient. The method includes acquiring an image of an affected area of a boney defect; reviewing the image and generating a diagnosis and a treatment plan to restore the boney defect; segmenting a region of the image having the boney defect; designing a crib to fit a boney defect area that is specific to the patient; printing a three-dimensional crib from a bioresorbable material; placing a particulate graft bioresorbable material in the crib; and fixating the crib to an overlying defect area to create the patient specific bone graft.

In another embodiment imaging of the patient's affected area is obtained. Said image is reviewed with appropriate image viewing software. A diagnosis is made by the surgeon and a treatment plan is created to restore the boney defect. Said image file is exported from the viewing software in a Digital Imaging in Communications of Medicine (DICOM) format and imported in a Computer Aided Design (CAD) software capable of reading the DICOM format. Once imported, the area of interest is segmented, or converted, into a 3-dimensional (3D) image in a Standard Tessellation Language (stl) format. This stl image can then be used to design and fabricate the patient specific bone graft. Options for designing the graft include, but are not limited, too, designing the graft in such a way as to approximate existing defect boundary structures and create a symmetric type appearance using the unaffected side of the body as a reference. In another option, the unaffected side can be mirrored to replace the boney defect. Once designed, the file is saved and ready to be bioprinted.

In the preferred embodiment, an image of the patient's oral facial area is obtained. The image is viewed with the image viewing software. A diagnosis is made by the doctor and a restoratively driven dental implant treatment plan is created to restore the patient's missing dentition. Dental implants act as anchors for dental prostheses. They require a certain width and height of patient jaw bone to ensure successful integration and function. The restoratively driven treatment plan is created as follows: pertinent physical exam, review of a patient's desires and expectations, review of imaging, acquisition and review of patient photographs and intraoral digital impressions. The imaging and intraoral digital impressions are aligned, or meshed, in dental CAD software to create a virtual representation of the area of interest. Once virtualized, the anticipated final restorations, which could be implant retained dentures, single unit implant crowns, or multiple unit implant bridges, are planned in ideal positions to provide optimal esthetic and functional results. Dental implants are then planned to act as anchors for the prostheses. Any bone deficiencies are then evaluated around the planned dental implant placement(s). If areas of boney deficiency are determined to be of a large size, a patient specific bone graft is designed to restore said deficient areas. Once designed, it may be printed via additive manufacturing.

Additive manufacturing may include methods such as any current 3D printing options. Specifically, the 3D designed graft may be 3D printed using an extrusion based bioprinter. A resorbable binding agent such as polycaprolactone (PCL) or polyglyconate is mixed with particulate allograft material in a ratio to obtain desired mechanical strength and preferred resorption time. Particulate allograft is bone harvested and processed from human cadavers. The particulate allograft may be comprised of cortical bone, cancellous bone, mineralized bone, demineralized bone, or a combination there of. Specifically, the size of the particulate allograft has to be of a size which allows extrusion of the combined binder and particulate out of a 18 or 19 gauge nozzle. This will allow the bioprinted graft to have an acceptable print size tolerance. Tight approximation between a bioprinted bone graft and it's implanted site ensures migration of the patient's cells into the graft allowing for successful integration and remodeling into mature native bone. Additional processing could be used in order to increase the surface porosity of the graft and also ensure sterility.

During the graft placement surgery, blood or bone marrow aspirate may be drawn from the patient. Blood may be processed in a manner to produce platelet rich fibrin (PRF) or platelet rich plasma (PRP.) The bioprinted graft is hydrated with either the PRF, PRP, or bone marrow aspirate to provide an osteoinductive feature. An additional osteogenic feature will be obtained if bone marrow aspirate is utilized. Once placed, the bioprinted graft is fixated with one or more fixation screws. Any border irregularities between the bioprinted graft and defect site may be filled with additional particulate graft particles. The graft is then covered with a non-resorbable or resorbable membrane for added protection prior to soft tissue closure and suturing.

In another embodiment, a bioprinter with multiple print heads can be used to print the patient specific bone graft with multiple materials. Xenograft particles can be used as the additional material. Xenograft bone is harvested from other animal species such as pig or cow. In one design, an outer layer of xenograft may be printed onto the patient specific bone graft. Adding this xenograft layer will decrease the resorption time of the graft, making it a more favorable option in some cases.

### BRIEF DESCRIPTION OF THE DRAWINGS

The application will become more fully understood from the following detailed description, taken in conjunction with the accompanying figures, wherein like reference numerals refer to like elements, in which:

FIG. 1. shows a schematic flow diagram of representative steps of the preferred embodiment.

FIG. 2A shows a screenshot of dental implant treatment planning.

FIG. 2A shows a screenshot of dental implant treatment planning.

FIG. 2B shows a cross-sectional view of the dental implant and prosthesis taken along the lines B-B in FIG. 2A.

FIG. 2C shows a view of the bone and dentition taken along the lines C-C in FIG. 2A.

FIG. 3 shows the 3D object of the bone defect in relation to the planned dental implant.

FIG. 4 shows the final design of the planned patient specific bone graft on the 3D object of the bone defect.

FIG. 5 shows the profile of the planned patient specific bone graft.

FIG. 6 shows the patient specific bone graft fixated to the patient's bone defect area.

FIG. 7A shows another embodiment of the bone graft.

FIG. 7B shows a resected jaw bone.

FIG. 7C shows a reconstructed jaw bone using the bone graft shown in FIG. 7A.

FIG. 8 shows a dual layer bioprinter patient specific bone graft.

FIG. 9 shows an exemplary crib for containment of bioresorbable material.

FIG. 10 shows the crib of FIG. 9 inserted on a boney defect of a patient's jaw.

### DETAILED DESCRIPTION OF THE EXEMPLARY EMBODIMENTS

Before turning to the figures which illustrate the exemplary embodiments in detail, it should be understood that the application is not limited to the details or methodology set forth in the following description or illustrated in the figures. It should also be understood that the phraseology and terminology employed herein is for the purpose of description only and should not be regarded as limiting.

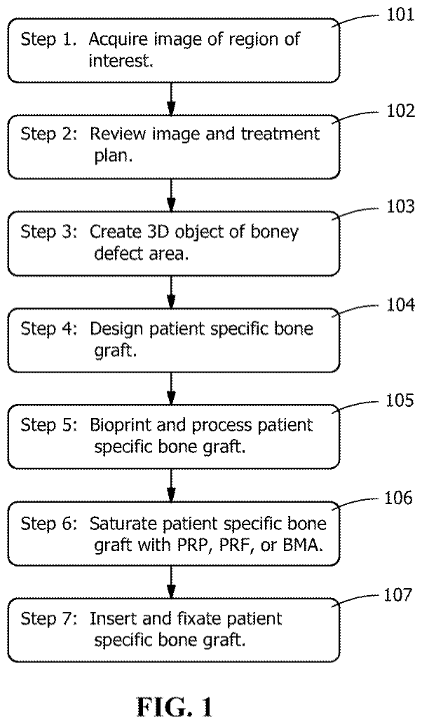

FIG. 1. Shows the schematic workflow for the preferred embodiment. An image of the desired area 201 may be acquired at step 101 with a computed tomography (CT) or cone beam computed tomography (CBCT) 202. The image is reviewed on a computer display by the doctor and a restoratively driven treatment plan 102 is developed to replace the patient's missing dentition with dental implant supported prostheses. The acquired image is saved as a Digital Imaging and Communications in Medicine (DICOM) file format and is imported into dental implant treatment planning software. Additional imaging, such as a digitally acquired impression of the patient's dental dentition may also be imported and aligned onto the DICOM image.

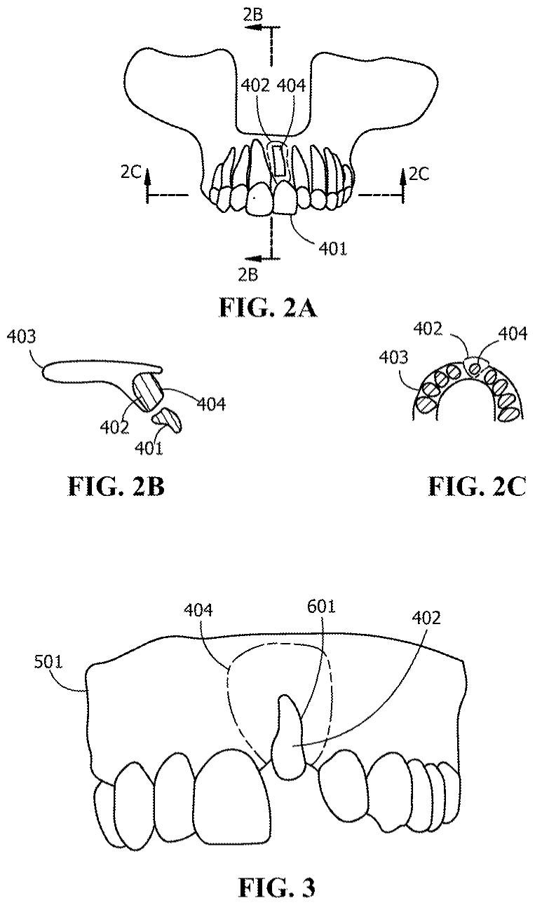

Referring to FIGS. 2A through 2C, a screenshot of dental implant treatment planning software is shown. First, the desired final prosthesis 401 (FIG. 2A) is designed to be in harmony and proper function with surrounding teeth. The dental implant 402 is planned in an ideal position to act as an anchor or attachment for said prosthesis. Views of the surrounding bone 403 are analyzed in relation to the planned dental implant 402 position (FIGS. 2B, 2C). A boney defect 404 is identified and judged to be a candidate for a patient specific bioprinted bone graft 701.

At this point, the region of the DICOM image with the boney defect 404 is segmented 103 with the dental implant treatment planning software. This segmentation process converts the image into a 3D object which now be saved as a Standard Tessellation Language (STL) file format. The dental implant 402 is also saved as a 3D object in STL format. The area of interest is then converted into three-dimensional object 501, e.g., an STL file format see, e.g., FIG. 3.

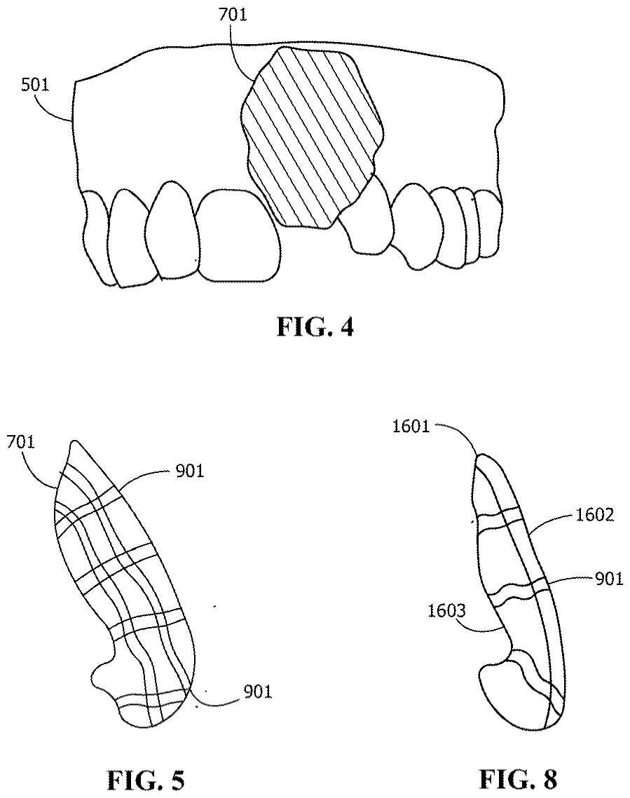

The 3D object 501 is now imported into computed aided design (CAD) software. FIG. 3 shows the dental implant 402 imported into its relative position to the 3D object 501 in the CAD software. The viewable portion of the dental implant 601, as well as the boney defect area 404, are used as references to design 104 the patient specific bone graft 701. Multiple tools are available in the CAD software to design, modify, and smooth the exterior surfaces of the designed patient specific bone graft 701 FIG. 4 shows the 3D object 501 with the designed patient specific bone graft 701. FIG. 5 shows a profile perspective of the designed patient specific graft 701. Additionally, the interior space or volume of the object may be modified to design hollow channels or conduits 901. These channels may facilitate vascular proliferation of the patient's blood supply allowing improved healing to take place. The designed patient specific bone graft 701 is now saved in STL file format. Examples of CAD software suitable for designing a graft include RealGuide and Materialise Mimics.

The patient designed specific bone graft 701 is imported into specialized software called slicing software. Slicing software is used to prepare 3d objects for 3D printing. Settings such as layer height, infill density, print speed, extrusion pressure, and heated bed temperature are adjusted according to ideal print conditions of the materials used in the print. The volume of the object to be printed is also calculated by the slicing software. This is used as a rough estimate to prepare the materials to be printed.

Biocompatible materials are used to print the patient specific bone graft. Polycaprolactone (PCL) powder is combined with particulate allograft, or alternatively, xenograft, in a specific ratio based upon prescribed graft properties such as resorption time and mechanical strength. Polyglyconate may be used instead of polycaprolactone. The two biocompatible materials are thoroughly mixed and loaded into a syringe that is inserted into a bioprinter (not shown), and is then used to print the bone graft 701. The print head heats the mixture into a liquid consistency before printing 105 commences. Once complete, the patient specific bone graft 701 is removed and undergoes post processing to ensure sterility. Another post processing step may be used to increase the surface area exterior of the patient specific bone graft. The patient specific bone graft is then packaged under sterile conditions and ready for use.

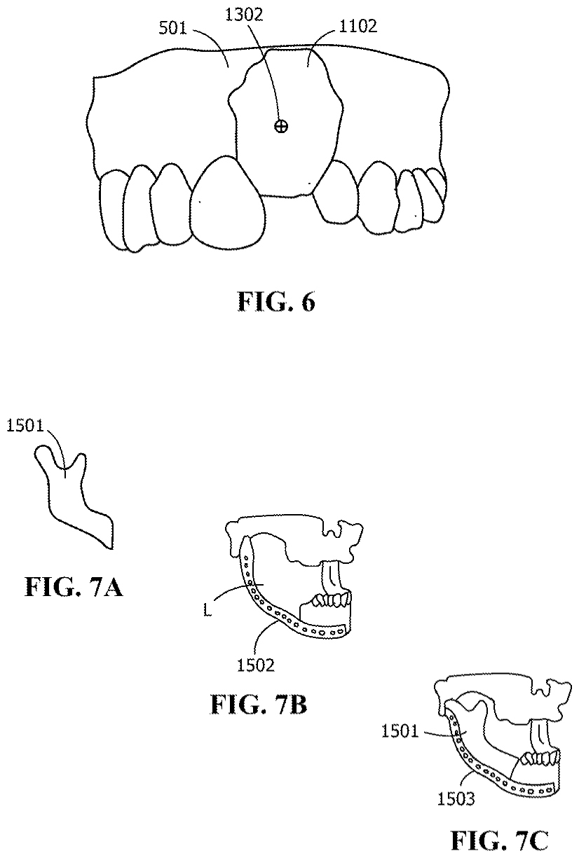

At the time of surgery venous blood or bone marrow aspirate is drawn from the patient. The venous blood is processed into platelet rich fibrin (PRF) or platelet rich plasma (PRP). The bone graft 701 is saturated 106 with either PRF, PRP, or bone marrow aspirate (BMA). FIG. 6 shows the boney defect area 501. The tissue over the boney defect area 501 is reflected and visualized by the surgeon. The boney defect area 501 is prepared to accept the graft. The patient specific bone graft 701 is seated and necessary adjustments are made to ensure close approximation between the surface of the boney defect and patient specific bone graft 701. The patient specific bone graft is fixated 107 with one or more surgical fixation screws 1302. Any irregularities between the patient specific bone graft and bone defect are filled with additional particulate graft material. A non-resorbable membrane may be sized and placed over the bone graft 701 to provide an additional layer of protection from soft tissue encroachment during the maturation phase of the graft. Alternatively, a resorbable membrane may be used instead of a non-resorbable membrane. The overlying tissue is then re-approximated and sutured closed in a tension free manner. Over a period of six to twelve months, the patient specific bone graft 701 will remodel into the patient's native bone and be ready for dental implant placement.

Referring next to FIGS. 7A through 7C, in an alternate embodiment, a patient specific bone graft 1501 (FIG. 7A) may be designed and fabricated in a similar manner as described previously to correct boney defects created by congenital malformations or tumor resections. FIG. 7B shows one such example where a portion of the jaw 1502 was resected due to oral cancer. Immediate reconstruction of the defect was not able to be performed at the time of surgery. A titanium plate 1503 (FIG. 7C) was placed to prevent gross malformation and tissue shrinkage. The missing portion of the jaw was designed using the intact side as a reference. The patient specific bone graft 1501 is printed and at the time of surgery fixated to the titanium plate with fixation screws.

In another embodiment, the patient specific bone graft is bioprinted in a dual layer technique. FIG. 8 shows a dual layer designed patient specific bone graft 1601. The patient specific bone graft is designed as mentioned previously, however, an additional outer surface layer 1602 is designed to be printed with a xenograft particulate/PCL mixture. The inner layer 1603, or layer to be in contact with the bone defect area, is printed with the allograft particulate/PCL mixture. Channels or conduits may also be designed in this embodiment. The ratio of xenograft to PCL may vary depending on the boney defect area to be reconstructed. In an embodiment a bioprinter with multiple print heads may be used to print the dual layer bone graft 1601. One print head is prepared with the PCL/allograft material and the other prepared with the PCL/xenograft material.

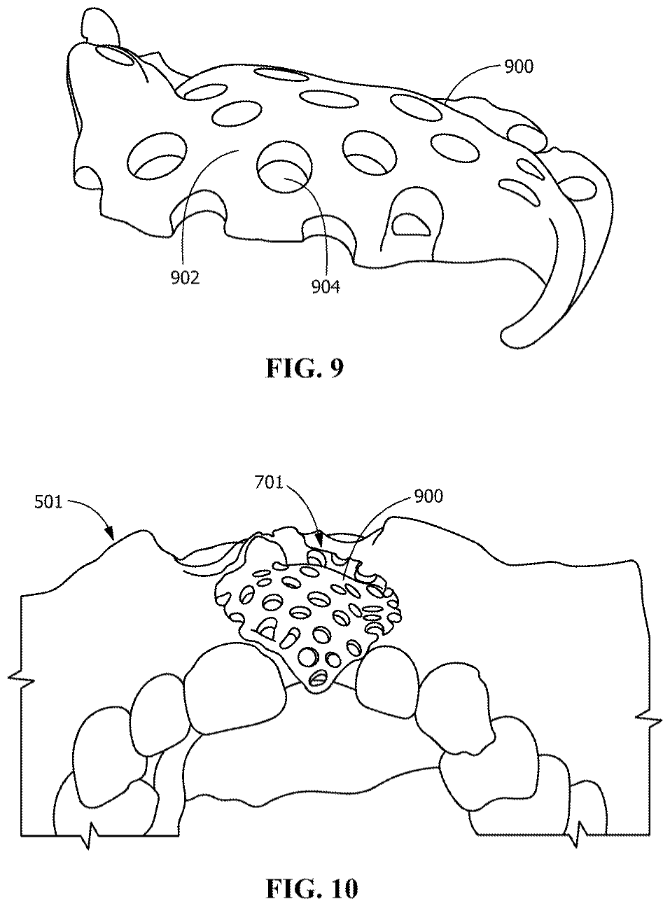

Referring next to FIGS. 9 and 10, in an embodiment the invention may include a crib 900 designed for and printed to fit a boney defect area that is specific to the patient. Crib 900 may be printed out of the same material as the graft. Crib 900 is a bioresorbable 3D containment structure having a contoured surface 902, and may optionally include perforations 904 to receive and shape the bioresorbable material. Bioresorbable material may include metal based alloys or polymers which may dissolve or be absorbed in the body. The surgeon places particulate graft bioresorbable material (not shown) in crib 900 and fixates the crib to the overlying defect area 404 to create the patient specific bone graft 701.

While the exemplary embodiments illustrated in the figures and described herein are presently preferred, it should be understood that these embodiments are offered by way of example only. Accordingly, the present application is not limited to a particular embodiment, but extends to various modifications that nevertheless fall within the scope of the appended claims. The order or sequence of any processes or method steps may be varied or re-sequenced according to alternative embodiments.

The present application contemplates methods, systems and program products on any machine-readable media for accomplishing its operations. The embodiments of the present application may be implemented using an existing computer processors, or by a special purpose computer processor for an appropriate system, incorporated for this or another purpose or by a hardwired system.

It is important to note that the construction and arrangement of the method for bioprinting patient specific bone grafts, as shown in the various exemplary embodiments is illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited in the claims. For example, elements shown as integrally formed may be constructed of multiple parts or elements, the position of elements may be reversed or otherwise varied, and the nature or number of discrete elements or positions may be altered or varied. Accordingly, all such modifications are intended to be included within the scope of the present application. The order or sequence of any process or method steps may be varied or re-sequenced according to alternative embodiments. In the claims, any means-plus-function clause is intended to cover the structures described herein as performing the recited function and not only structural equivalents but also equivalent structures. Other substitutions, modifications, changes and omissions may be made in the design, operating conditions and arrangement of the exemplary embodiments without departing from the scope of the present application.

As noted above, embodiments within the scope of the present application include program products comprising machine-readable media for carrying or having machine-executable instructions or data structures stored thereon. Such machine-readable media can be any available media which can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions comprise, for example, instructions and data which cause a general purpose computer, special purpose computer, or special purpose processing machines to perform a certain function or group of functions.

It should be noted that although the figures herein may show a specific order of method steps, it is understood that the order of these steps may differ from what is depicted. Also two or more steps may be performed concurrently or with partial concurrence. Such variation will depend on the software and hardware systems chosen and on designer choice. It is understood that all such variations are within the scope of the application. Likewise, software implementations could be accomplished with standard programming techniques with rule based logic and other logic to accomplish the various connection steps, processing steps, comparison steps and decision steps.

1. A method for bioprinting a three-dimensional bone graft for a patient comprising:
acquiring an image of an affected area of a boney defect;
reviewing the image and generating a diagnosis and a treatment plan to restore the boney defect;
segmenting a region of the image having the boney defect;
designing a bone graft based on the boney defect;
printing the bone graft using a biomaterial;
obtaining a platelet based composition derived from a patient undergoing the implant treatment;
saturating the bone graft with the platelet based composition;
inserting the bone graft; and
fixating the bone graft to the affected area.

2. The method of claim 1, further comprising:
converting the image in a Digital Imaging in Communications of Medicine [DICOM] format; and
exporting the converted DICOM image to a Computer Aided Design (CAD) software capable of reading a DICOM formatted image.

3. The method of claim 2, further comprising acquiring a digital impression of a dental dentition of the patient;
and aligning the dental dentition onto the converted DICOM image.

4. The method of claim 1, wherein generating further comprises performing the treatment plan using a treatment planning software program; and
exporting the image from the viewing software in a DICOM format and receiving the DICOM formatted image in a CAD software program; the CAD software program configured for reading the DICOM format.

5. The method of claim 1, further comprising converting an image of the affected area into a 3-dimensional (3D) image in a Standard Tessellation Language (stl) format; and
fabricating the bone graft using the stl formatted image.

6. The method of claim 1, further comprising designing the bone graft to approximate one or more existing defect boundary structures and creating a symmetric appearance using an unaffected side of a patient's anatomy as a reference.

7. The method of claim 1, further comprising mirroring an unaffected side of the patient to replace the boney defect.

8. The method of claim 1, wherein the step of segmenting comprises:
converting the image into a 3D object and saving the three-dimensional object as a Standard Tessellation Language (STL) file format; and
saving an image of a dental implant as a 3D object in the STL format.

9. The method of claim 1, further comprising bioprinting a biocompatible material in combination with a particulate graft tissue in a ratio based upon at least one graft properties selected from a resorption time and a mechanical strength; and loading the combined biocompatible material and the particulate graft tissue into a bioprinter syringe; wherein the graft tissue comprises one of allograft and xenograft.

10. The method of claim 8, wherein the biocompatible material comprises polycaprolactone.

11. The method of claim 8, wherein the biocompatible material comprises polyglyconate.

12. The method of claim 8, further comprising:
drawing a quantity of venous blood from the patient;
processing the quantity of venous blood into a platelet rich fibrin (PRF); and
saturating the bone graft with the PRF.

13. The method of claim 8, further comprising:
drawing a quantity of venous blood from the patient;
processing the quantity of venous blood into a platelet rich plasma (PRP); and
saturating the bone graft with the PRP.

14. The method of claim 8, further comprising
drawing a quantity of bone marrow aspirate from the patient; and
saturating the bone graft with the bone marrow aspirate.

15. The method of claim 1, further comprising:
reflecting a tissue over the boney defect area; and
visualizing the reflected tissue;
preparing the boney defect area to accept the bone graft;
seating the bone graft;
adjusting the bone graft to ensure close approximation between the boney defect and patient specific bone graft.

16. The method of claim 1, wherein the step of fixating comprises fixating the bone graft with one or more surgical fixation screws.

17. The method of claim 1, further comprising:
filling one or more irregularities between the patient specific bone graft and bone defect with a particulate graft material.

18. The method of claim 1, further comprising placing a membrane over the bone graft.

19. The method of claim 1, wherein the membrane is a non-resorbable membrane or a resorbable membrane.

20. A method for bioprinting a three-dimensional bone graft for a patient comprising:
acquiring an image of an affected area of a boney defect;
reviewing the image and generating a diagnosis and a treatment plan to restore the boney defect;
segmenting a region of the image having the boney defect;
designing a crib to fit a boney defect area that is specific to the patient;
printing a three-dimensional crib from a bioresorbable material;
placing a particulate graft bioresorbable material in the crib; and
fixating the crib to an overlying defect area to create the patient specific bone graft.
